SEQUENCE LISTING (17 sequences)

SEQ ID NO: 1 - protein, 105 aa, Ichthyophthirius multifiliis
  MUTAGEN (2): A/S WHERE S HAS BEEN DERIVED FROM THE SYNTHETIC GENE.
  MUTAGEN (4)..(105): Q - THE GLUTAMINE CODONS TAA AND TAG IN THE ORIGINAL SEQUENCE, HAVE BEEN REPLACED WITH THE UNIVERSAL GLUTAMINE CODONS CAG OR CAA IN THE SYNTHETIC GENE
  MUTAGEN (34): V/G WHERE G HAS BEEN DERIVED FROM THE SYNTHETIC GENE.
  MUTAGEN (105): V/I WHERE I HAS BEEN DERIVED FROM THE SYNTHETIC GENE.
GAAQGEANGNQPFAANNAARGICVPCQINRVGSVTNAGDLATLATQCSTQCPTGTALDDGVTDVFDRSAAQCVKCKPNFYYNGGSPQGEAPGVQVFAAGAAAAGV

SEQ ID NO: 2 - DNA, 316 bp, Ichthyophthirius multifiliis
  CDS (1)..(315)
ggtgctgcttaaggagaagctaatggtaattaacctttcgcagcaaataatgctgctagaggtatatgtgtaccatgccaaataaacagagtaggctctgttaccaatgcaggtgacttagctactttagccacataatgcagtacttaatgtcctactggcactgcacttgatgatggagtgacagatgtttttgatagatcagccgcataatgtgttaaatgcaaacctaacttttactataatggtggttctccttaaggtgaagctcctggcgtttaagtttttgctgctggtgctgccgctgcaggtgttg

SEQ ID NO: 3 - protein, 6 aa, Ichthyophthirius multifiliis
GEANGN

SEQ ID NO: 4 - protein, 34 aa, Ichthyophthirius multifiliis
PFAANNAARGICVPCQINRVGSVTNAGDLATLAT

SEQ ID NO: 5 - protein, 20 aa, Ichthyophthirius multifiliis
CPTGTALDDGVTDVFDRSAA

SEQ ID NO: 6 - protein, 15 aa, Ichthyophthirius multifiliis
CVKCKPNFYYNGGSP

SEQ ID NO: 7 - protein, 6 aa, Ichthyophthirius multifiliis
GEAPGV

SEQ ID NO: 8 - protein, 11 aa, Ichthyophthirius multifiliis
VFAAGAAAAGV

SEQ ID NO: 9 - DNA, 316 bp, Ichthyophthirius multifiliis
  CDS (1)..(315)
ggatccgctcagggagaagctaatggtaatcagcctttcgcagcaaataatgctgctagaggtatatgtgtaccatgccaaataaacagagtaggctctggtaccaatgcaggtgacttagctactttagccacacaatgcagtactcagtgtcctactggcactgcacttgatgatggagtgacagatgtttttgatagatcagccgcacagtgtgttaaatgcaaacctaacttttactataatggtggttctcctcagggtgaagctcctggccttcaggtttttgctgctggtgctgccgctgcaggaattc

SEQ ID NO: 10 - protein, 105 aa, Ichthyophthirius multifiliis
GSAQGEANGNQPFAANNAARGICVPCQINRVGSGTNAGDLATLATQCSTQCPTGTALDDGVTDVFDRSAAQCVKCKPNFYYNGGSPQGEAPGLQVFAAGAAAAGI

SEQ ID NO: 11 - DNA, 66 bp, Ichthyophthirius multifiliis
ggcggatccgctcagggagaagctaatggtaatcagcctttcgcagcaaataatgctgctagaggt

SEQ ID NO: 12 - DNA, 60 bp, Ichthyophthirius multifiliis
accggtaccagagcctactctgtttatttggcatggtacacatatacctctagcagcatt

SEQ ID NO: 13 - DNA, 66 bp, Ichthyophthirius multifiliis
accggtaccaatgcaggtgacttagctactttagccacacaatgcagtactcagtgtcctactggc

SEQ ID NO: 14 - DNA, 59 bp, Ichthyophthirius multifiliis
cgtgatctatcaaaaacatctgtcactccatcatcaagtgcagtgccagtaggacactg

SEQ ID NO: 15 - DNA, 68 bp, Ichthyophthirius multifiliis
cctgatcagccgcacagtgtgttaaatgcaaacctaacttttactataatggtggttctcctcagggt

SEQ ID NO: 16 - DNA, 69 bp, Ichthyophthirius multifiliis
gcgaattcctgcagcggcagcaccagcagcaaaaacctgaacgccaggagcttcaccctgaggagaacc

SEQ ID NO: 17 - DNA, 17 bp, Ichthyophthirius multifiliis
tagcatggcctttgcag

(19) United States
(12) Patent Application Publication
(10) Pub. No.: US 2007/0071776 A1
(43) Pub. Date: Mar. 29, 2007

(54) Recombinant Vaccine Against Fish Infectious Diseases

(75) Inventors: Yoke Min SIN, Singapore (SG); Toong Jin LAM, Singapore (SG); Zhiyuan GONG, Singapore (SG)

Correspondence Address: LOWE HAUPTMAN BERNER, LLP, 1700 DIAGONAL ROAD, SUITE 300, ALEXANDRIA, VA, 22314, US

(73) Assignee: NATIONAL UNIVERSITY OF SINGAPORE, Singapore (SG)

(21) Appl. No.: 11/530,169

(22) Filed: Sep. 8, 2006

Related U.S. Application Data
(63) Continuation of application No. 09/196,161, filed on Nov. 20, 1998, now abandoned.

(30) Foreign Application Priority Data
Sep. 28, 1998 (SG) 9803188-3

(51) Int. Cl. A61K 48/00; A61K 39/00

(52) U.S. Cl. 424/269.1; 514/44

(57) ABSTRACT

A recombinant vaccine that is useful for immunizing fish against ciliated ectoparasitic protozoans that cause white spot disease also known as ‘ich’. The disease is caused by Ichthyophthirius multifiliis which is a ciliated ectorparasitic protozoan that is an obligate parasite and therefore cannot grow in vitro. The instant vaccine replaces chemotherapy and vaccines prepared from I. Multifiliis production in live fish. The effective element of the vaccine is a recombinant fusion protein derived from an artificial DNA sequence for immobilization antigen, repeat I, of Ichthyophthirius multifiliis. The novel DNA nucleotide and amino acid sequences are disclosed.

### RELATED APPLICATIONS

The present application is a continuation of application Ser. No. 09/196,161, filed Nov. 20, 1998, and is based on, and claims priority from, SG Application Number 9803188-3, filed Sep. 28, 1998.

### TECHNICAL FIELD

This invention is directed to a recombinant vaccine for immunizing fish against white spot disease (also known as ich ).

### BACKGROUND OF THE INVENTION

White spot disease is a severe infectious disease that adversely affects warm water fish. . It is caused by a ciliated ectoparasitic protozoan, Ichthyophthirius multifiliis that causes high morbidity and mortality in many freshwater fish. In the past, when fish show clinical signs of infection by this disease, they have commonly been treated with chemicals (Cross, D. G., “A REVIEW OF METHODS TO CONTROL ICHTHYOPHTHIRIASIS”, Prog. Fish-culture, 34, (1972) 165-169; Gratzek, J. B., “CONTROL AND THERAPY OF FISH DISEASES”, Advances Vet. Sci. Comparat. Med., 27, (1983) 297-324; Post, G., “TEXTBOOK OF FISH HEALTH” (Revised and expanded edition), T.F.H. Publications Inc, USA, 1987). However, these chemicals usually leave residues in the fish tissues that can endanger not only the health of the fish, but they can also adversely affect humans who are the ultimate consumers of the infected fish.

On the other hand, protection of many fish species against Ichthyophthiriasis has been reported through the use of immunization with I. Multifiliis. Such reports of immunized fish include:

goldfish (Ling, K. H., Sin, Y. M. and Lam, T. J., “PROTECTION OF GOLDFISH AGAINST SOME COMMON ECTOPARASITIC PROTOZOANS USING ICHTHYOPHTHIRIUS MULTIFILIIS AND TETRAHYMENA PYRIFORMIS FOR VACCINE”, Aquaculture, 116 (1993) 303-314;

mirror carp (Hines, R. S. and Spira, D. T., “ICHTHYOPHTHIRIASIS IN THE MIRROR CARP CYPRINUS CARPIO (L), IV. PHYSIOLOGICAL DISFUNCTION”, J. Fish Biol., 6 (1974) 365-371;

channel catfish (Gover, B. A., Dawe, D. L. and Gratzek, B., “PROTECTION OF CHANNEL CATFISH, ICTALURUS PUNCTATUS RAFINESQUE, AGAINST I. MULTIFILIIS FOUQUET BY IMMUNIZATION”, J.Fish Biol., 17 (1980) 311-316;

rainbow trout (Wolf, K. and Markiw, M. E., “ICHTHYOPHTHIRIASIS: IMMERSION IMMUNIZATION OF RAINBOW TROUT (SALMO GAIRDNERI) USING TETRAHYMENA THERMOPHILA AS A PROTECTIVE IMMUNOGEN”, Can. J. Fish Aquat. Sci., 39 (1982) 1772-1725;

tilapia (Subasinghe, R. P. and Sommerville, C., “ACQUIRED IMMUNITY OF OREOCHROMIS MOSSAMBISCUS TO THE CILIATE ECTOPARASITE ICHTHYOPHTHIRIUS MULTIFILIIS (FOUQUET)” In: J. L Maclean, L. B. Dizon and L. V. Osillos (editors), The first Asian Fisheries Forum, Asean Fisheries Society, Manila, Philippines, 1987, 279-283); and

some other common ornamental fish (Ling K. H., Sin Y. M. and Lam T. J., “A NEW APPROACH TO CONTROLLING ICHTHYOPHTHIRIASIS IN A CLOSED CULTURE SYSTEM OF FRESHWATER ORNAMENTAL FISH”, J. fish Dis., 14 (1991) 595-598).

Until now, no known methods have been reported as to how to in vitro grow the protozoan, I. Multifiliis, that has caused the problem. The vaccines (whole cells, cell fragments or extracts of the pathogen), that have been prepared and used by different laboratories, have only been obtained from an infected live fish since the offending protozoan is an obligate parasite. Preparation of such vaccines from live fish is very time-consuming, costly and impractical.

An antigenic relationship has been demonstrated between I. Multifiliis and Tetrahymena pyriformis, a free-living and cultivable non-pathogenic ciliated protozoan (Goven , B. A., Dawe, D. L. and Gratzek, J. B., “PROTECTION OF CHANNEL CATFISH, ICTALURUS PUNCTATUS RAFINESQUE, AGAINST ICHTHYOPHTHIRIUS MULTIFILIIS FOUQUET BY IMMUNIZATION”, J. Fish Biol., 17 (1980) 311-316). These workers suggested that T. pyriformis could be used as a vaccine for inducing protection against Ichthyophthiriasis (Goven,B. A., Dawe, D. L and Gratzek, J. B., “IN VITRO DEMONSTRATION OF SEROLOGICAL CROSS-REACTIVITY BETWEEN ICHTHYOPHTHIRIUS MULTIFILIIS FOUQUET AND T. PYRIFORMIS LWOFF”, Dev. Comp. Immunol., 5 (1981) 283-289). A ciliary protein extracted from T. pyriformis was shown to be an effective vaccine against the disease caused by I. Multifiliis in freshwater fish and Cryptocryon in marine fish (U.S. Pat. No. 4,309,416). The presence of an immobilization antigen (i-Ag) in I. Multifiliis was reported by Dickerson, H. W., Clark,T. G. and Findly, R. C., “ICHTHYOPHTHIRIUS MULTIFILIIS: HAS MEMBRANE-ASSOCIATED IMMOBILIZATION ANTIGENS”, J.Protozool., 36 (1989)159-164) and by Lin,T. L. and Dickerson, H. W., “PURIFICATION AND PARTIAL CHARACTERIZATION OF IMMOBILIZATION ANTIGEN FROM ICHTHYOPHTHIRIUS MULTIFILIIS”, J. Protozool., 39 (1992) 457-463).

A partial cDNA clone for 48 kDa i-Ag was isolated from a cDNA library and the amino acid sequence that was deduced from the cDNA clone contained three tandem repeats (Clark, T. G., McGraw, R. A. and Dickerson, H. W., “DEVELOPMENTAL EXPRESSION OF SURFACE ANTIGEN GENES IN THE PARASITIC CILIATE ICHTHYOPHTHIRIUS MULTIFILIIS”, Proc. Natl. Acad. Sci. USA, 89 (1992) 6363-6367). In fact, the degree of immune protection was correlated with the levels of antibody titers against this immobilization antigen in the plasma and skin mucus of the fish that had been immunized against I. Multifiliis (Clark , T. G., Dickerson, H. W., and Findly, R. C., “IMMUNE RESPONSE OF CHANNEL CATFISH TO CILIARY ANTIGENS OF ICHTHYOPHTHIRIUS MULTIFILIIS”, Devel. Comp. Immunol.,12 (1988)581-594 and T. pyriformis (Ling, K. H., Sin, Y. M. and Lam T. J., “PROTECTION OF GOLDFISH AGAINST SOME COMMON ECTOPARASITIC PROTOZOANS USING ICHTHYOPHTHIRIUS MULTIFILIIS AND TETRAHYMENA PYRIFORMIS FOR VACCINATION”, Aquaculture, 116 (1993) 303-314). Fish immunized with I. Multifiliss and T. pyriformis by immersion and intraperitoneal injection developed protective immunity not only against I. Multifiliis, but also against other ciliated ectoparasitic protozoans that are commonly found in the tropics (Ling, K. H., Sin, Y. M. and Lam T. J., “PROTECTION OF GOLDFISH AGAINST SOME COMMON ECTOPARASITIC PROTOZOANS USING ICHTHYOPHTHIRIUS MULTIFILIIS AND TETRAHYMENA PYRIFORMIS FOR VACCINATION”, Aquaculture, 116 (1993) 303-314.

Since the chemicals that have been found to be effective against these adverse ciliated protozoans are not approved for use in food fish by the USDA, and many other countries in the world, immunization may be the only practical approach to controlling this white spot disease. This can be done by either direct immunization of the fish with vaccines, using whole cells or ciliary protein extracts from I.multifilis, as discussed above or by passive protection using immobilization antibodies against the pathogen released by the immunized fish in a closed culture system (U.S. Pat. No. 5,643,571).

### OBJECTS AND BRIEF DESCRIPTION OF THE INVENTION

An important object of this invention is to provide an effective vaccine for immunization against infectious fish ectoparasitic protozoan, Ichthyophthirius mutifiliis (He, J., Yin, Z., Xu, G., Gong, Z., Lam, T. J. and Sin, Y. M., “PROTECTION OF GOLDFISH AGAINST ICHTHYOPHTHIRIUS MULTIFILIIS BY IMMUNIZATION WITH A RECOMBINANT VACCINE”, Aquaculture, 158(1997)1-10) that has been produced by recombinant techniques, since no vaccine has as yet been obtained, nor is it likely that one can be obtained, using in vitro systems.

Another object of this invention is to provide an effective vaccine for the immunizaton of fish, not only against Ichthyophthirius mutifiliis, but also against other, taxonomically related, ciliated ectoparasitic protozoans, by recombinant techniques.

A further object of this invention is to provide a feasible method for commercial production of this vaccine.

Other and additional objects will become apparent from a consideration of this entire specification including the claims appended hereto.

In accordance with and fulfilling these objects, one aspect of this invention is a vaccine against ciliated protozoan infection that has been prepared by recombinant techniques. A gene fragment, containing a complete repeat I of I. Multifiliis, was synthesized and assembled based on the amino acid sequence of i-AgI. The recombinant i-AgI was expressed in bacteria, as a glutathione S-transferase (GST-iAgI) fusion protein. The fusion protein was purified from bacterial lysate and successfully used as a vaccine for immunization. The modified sequences according to this invention can be used to produce the recombinant fusion protein by any organism. These are exemplifed by: yeast, baculovirus in insect cells, transgenic mammals, other vertebrate and invertebrate and transgenic plants using universal genetic codons. These universal codons are CAG and CAA, which are glutamine codons.

### BRIEF DESCRIPTION OF THE DRAWING

FIG. 1A is an illustration of the epitope of i-Ag included in a GST-iAgI recombinant protein;

FIG. 1B is an illustration of six synthetic oligonucleotides that were used to construct the i-AgI epitope region;

FIG. 1C is a diagram of the plasmid pGST-iAgI that was used for the production of GST-iAgI protein;

FIGS. 2(A) is a plan view of a gel electrophoresis of bacterial expressions of GST-iAgI fusion protein; and

FIG. 2(B) is a plan view of a Western Blot analysis.

### DETAILED DESCRIPTION OF THE INVENTION

A 316 bp gene fragment containing an antigenic epitope of the 48K immobilization antigen of Ichthyophthirius multifiliis (iAgI) was assembled from six synthetic oligonucleotides and cloned into a bacterial expression vector pGEX2T. The gene construct was introduced into Escherichia coli and the glutathione S-transferase-iAgI (GST-iAgI) fusion protein was successfully expressed. Antisera against GST-iAgI fusion protein from catfish showed a positive reaction with a tomite protein of about 48K, suggesting that the recombinant protein contains an antigenic epitope of i-AgI. Naive goldfish, that were immunized with purified GST-iAgI fusion protein, were challenged with a lethal dose of infectious tomites of I. Multifiliis. The results showed that the recombinant GST-iAgI fusion protein can be used as a vaccine against the infection of not only I. Multifiliis but also against diseases caused by other taxonomically similar pathogens, that is the cilated ectoparasitic protozoans.

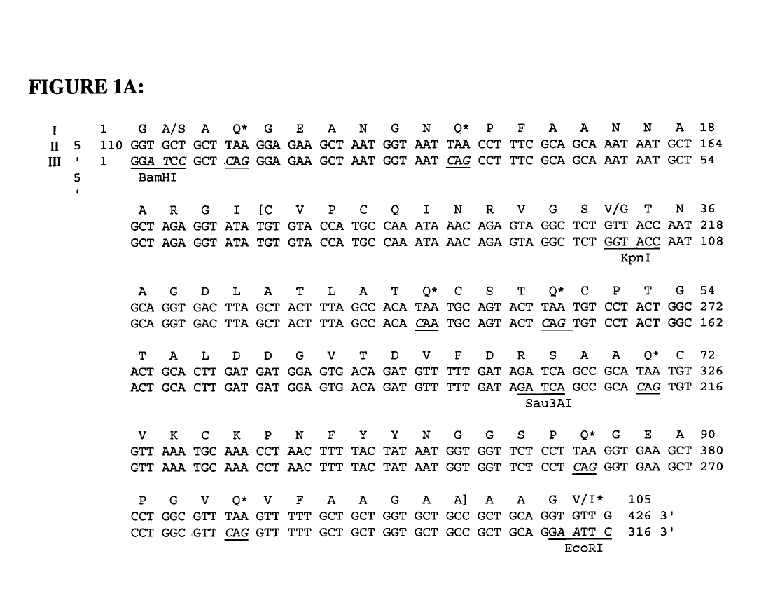

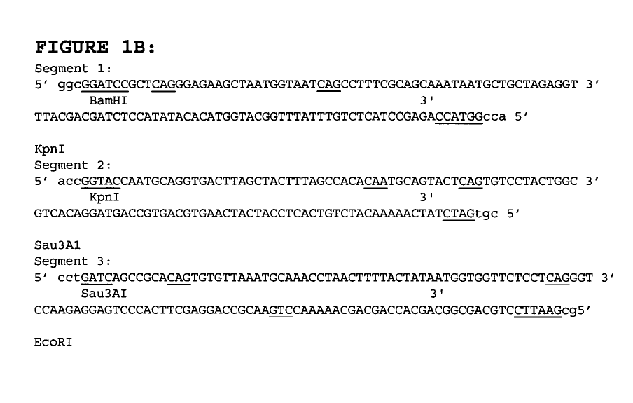

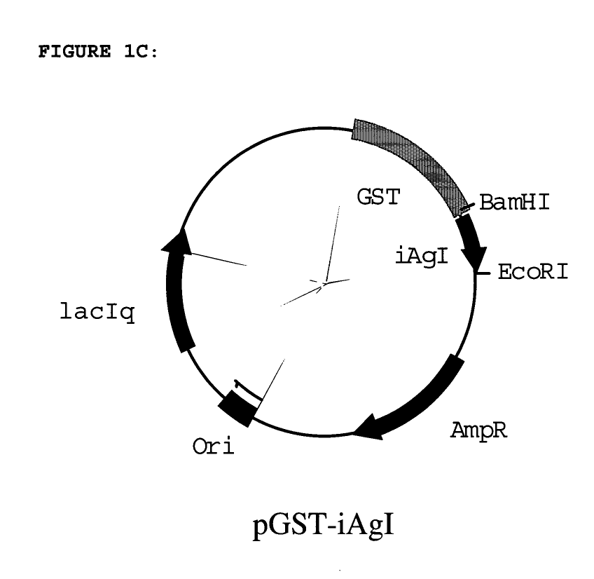

Referring now to FIG. 1(A), the amino acid sequence of the epitope of i-Ag is set forth on line I, the corresponding nucleotide sequences from the original i-Ag cDNA clone are set forth on line II, and the synthetic DNA fragment of this invention is set forth on line III. This Fig. aligns these three sequences. Two conserved amino acid substitutions between the original and the synthetic i-AgI DNAs have been indicated as A/S and V/G, where S and G have been derived from the synthetic gene. The amino acids from and including 23 to 101 have been marked off by square brackets. These are a repeat of i-Ag (see Clark et al., 1992, above). The glutamine codons TAA and TAG in the original sequence, which are the universal codons, are indicated by asterisks, and have been replaced with the universal glutamine codons CAG and CAA (underlined) in the synthetic gene. The restriction sites in the synthetic gene that are used for cloning have also been underlined. The changed nucleotides for the creation of the restriction enzyme sites, as well as for the replacement of the glutamine codons, have been shown in italics.

Referring now to FIG. 1(B), there is shown six (6) synthetic oligonucleotides that were used to construct the i-AgI epitope region. There are three (3) DNA segments, each of which is constructed with two (2) oligonucleotides that were derived from the coding strand (upper) and the non-coding strand (lower). The 3′ complimentary nucleotides (12bp) have been aligned, and the 5′ restriction sites and changed glutamine codons have been underlined.

Referrring now to FIG. 1(c), there is shown a diagram of the plasmid pGST-iAgI used for the production of GST-iAgI protein. The 316 bp synthetic DNA fragment was inserted into pGEX2T vector between BamHI and EcoRI sites as described herein.

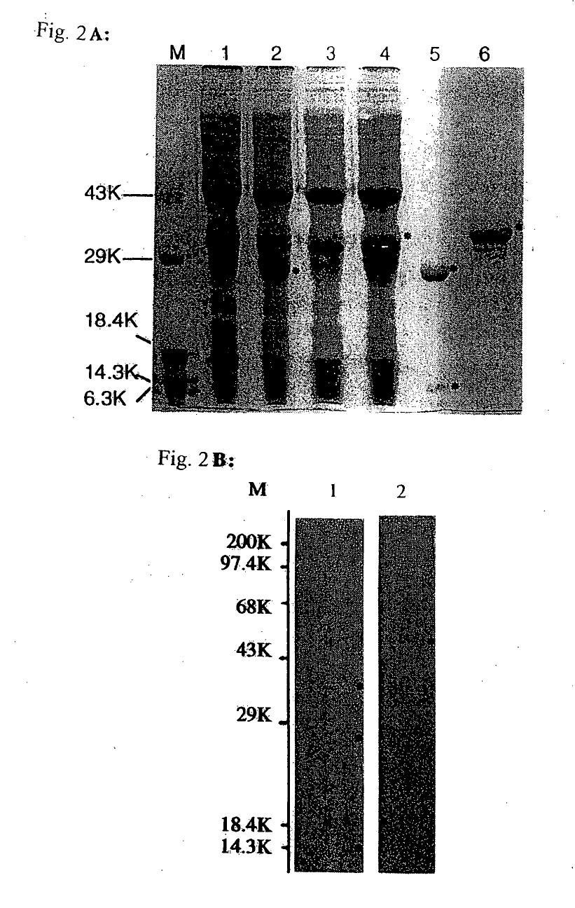

Referring now to FIG. 2(A), there are shown bacterial expressions of GST-iAgI fusion protein. Lanes 1-4 show total lysate of bacteria transformed with pGEX2T (lanes 1 and 2), or with pGST-iAgI (lanes 3 and 4). Lanes 1 and 3 had no IPTG induction, and lanes 2 and 4 had IPTG induction. Lanes 5 shows purified GST-iAgI with thrombin digestion and lane 6 shows GST-iAgI without thrombin digestion. The GST-iAgI was cleaved into two (2) parts, as indicated. The SDS-PAGE was stained with coomassie brilliant blue. The position of the protein is indicated by the solid dot on the right of each lane. M was a prestained protein standard (Gilbco, Life Sciences) in KDa.

Referring now to FIG. 2(B), which is a Western Blot analysis, in lane 1 there is shown GST-iAgI fusion protein that has been partially digested with thrombin. The solid dots indicate intact fusion protein GST and i-AgI. In lane 2 there is shown the Tomite total protein. M indicates a high range of prestained protein standard (Gilbco, Life Sciences) in KDa.

Materials and Methods

Design, Synthesis and Assembly of The ‘iAGI’ GENE

The immobilization antigen of I. Multifiliis (i-AgI) consisted of three tandem repeats in its central region. Three DNA segments of similar length were designed to cover a complete repeat I of i-AgI. For each segment, two oliogonucleotides, representing both coding and noncoding strands, were synthesized. To ensure efficient intermolecular annealing, the two oligonucleotides contained 12 complementary nucleotides at their 3′ ends (FIG. 1B). Restriction sites were engineered at the 5′ ends of each oligonucleotide to facilitate cloning. To generate double-stranded DNA from the oligonucleotides, 10 μl each of the corresponding two oligonucleotides were mixed, at the same concentration (1 μg/μl), with 5 μl of 10 ×Klenow buffer (500 mM Tris, pH7.2, 100 mM MgSO₄, 1 mM dithiothreitol) and 18 μl distilled water. The reaction mixture was heated and maintained at 80° C. for 5 min and then allowed to cool to 37° C., followed by addition of 5 μl of 2.5 mM deoxyribonucleotides (dNTP) and 2 μl of Klenow fragment (Promega) to initiate DNA synthesis at room temperature. After 40 minutes EDTA was added to a final concentration of 10 mM to stop the reaction,and this was followed by incubation at 75° C. for 5 min to inactivate the Klenow enzyme. The double-stranded DNA segments were purified by phenol/chloroform extraction and ethanol precipitation, and dissloved in 50 μl of distilled water. To assemble the three segments into a longer DNA fragment, half of double-stranded segments 1, 2 and 3 were double digested with BamHI-KpnI, KpnI-Sau3A1 and Sau3A1-EcoRI, respectively. The digested segments were recovered by gel purification, using low-melting-temperature agarose, and dissloved in 25 μl of water. About 0.1 μg of digested segment 1 was ligated into 0.1 μg of pBluescript vector (stratagene) that had been predigested with BamHI and KpnI. The ligation was performed overnight at 4° C. in a 20 μl reaction mixture. Similarly, 0.2 μg each of segment 2 and segment 3 were ligated together into pBluescript vector (Stratagene) that had been predigested with KpnI and EcoRI. 10 μl of each ligation reaction mixture was transferred into 100 μl of E. coli competent cell (XL1-Blue strain, Startagene) according to the standard protocol described by Sambrook, J., Fritsch E. F. and Maniatis,T. (“MOLECULAR CLONING, A LABORATORY MANUAL”2^{nd} edition, Cold Spring Harbor Laboratory Press, New York, 1989). The resulted plasmids were isolated with Qiagen Plasmid Isolation Kit (Qiagen) and sequenced from both ends for confirmation. The verified segment 1 and segments 2 and 3 were removed from pBluescript by digestion with BamHI-KpnI and KpnI-EcoRI respectively. 0.1 μg of verified segment 1 and segments 2 and 3 each were ligated into 0.1 μg of pGEX2T (Pharmacia) vector cut with BamHI and EcoRI. The volume of 10 μl ligation reaction was transformed into E. coli (XL1-Blue strain, Stratagene), and the resulting construct was called pGST-iAgI. Its sequence was confirmed again by sequencing with the primer 5′-TAGCATGGCCTTTGCAG-3′, upstream of the cloning site of pGEX2T vector.

Expression and Purification of GST Fusion Protein

An overnight culture of E. coli harboring pGST-iAgI was diluted to 1:20 in fresh LB medium containing 50 μg/ml ampicillin and grown at 37° C. with vigorous shaking. Isopropyl 1-thio-β-D-galactoside (IPTG) was added to a final concentration of 0.1 mM when the OD₆₀₀ reached about 0.5. Bacteria were harvested, by centrifugation at 2,000 G at 4° C. for 15 min, 3 hours after the addition of IPTG. The bacteria were resuspended in 300 μl of phosphate-buffered saline (PBS), boiled in SDS-sample buffer for 5 min and analyzed by electrophoresis (according to Laemmli, U. K., “CLEAVAGE OF STRUCTURE PROTEIN DURING THE ASSEMBLY OF THE HEAD OF BACTERIOPHAGE T4”Nature, 227(1970) 680-685)) using an SDS-polyacrylamide gel.

The GST-iAgI fusion protein was purified using Glutathione Sepharose 4B Beads (Pharmacia) as previously described (Gong, Z. and Hew, C. L., “ZINC AND DNA BINDING PROPERTIES OF A NOVEL LIM HOMEODOMAIN PROTEIN ISL-2”, Biochemistry, 33 (1995)15149-15158). Shortly after IPTG induction, 1 liter of bacterial culture was harvested by centrifugation and resuspended in 10 ml of lysis buffer (50 mM Tris, pH8.0, 0.1M NaCl, 1 mM EDTA), to which lysozyme had been added to a final concentration of 5 mg/ml. After incubation at room temperature for 5 min, bacteria, that were lysed with 1% Triton-100. MgCl₂ and DNase I, were then added, in amounts sufficient to produce final concentrations of 5 mM and 10 μg/ml respectively, to digest viscous DNA. The lysate was cleared by centrifugation. The supernatant was incubated with 1 ml of a 50% slurry of Glutathione Sepharose 4B Beads (Pharmacia) at room temperature for 1 hour with gentle agitation. The beads were then washed three times in 10× of bed volume of PBS. The GST-iAgI fusion protein was eluted from beads with 15 mM reduced glutathione (Sigma). The concentration of purified fusion protein was measured using Bio-Rad Protein Assay (Bio-Rad). Thrombin (Sigma) digestion of purified pGST-iAgI fusion protein was carried out according to the method described by Smith, D. B. and Johnson, K. S. “SINGLE-STEP PURIFICATION OF POLYPEPTIDES IN ESCHERICHIA COLI AS FUSION GLUTATHIONE S-TRANSFERASE”Gene, 67 (1988)31-40.

Analysis of Antigenccty of GST-IAGI Fusion Protein by Western Blot.

Catfish, of 20-25 cm in length, were immunized by intraperitoneal injection of 200 μg of GST-iAgI fusion protein mixed with Freund's complete adjuvant (FCA). A similar dose of the fusion protein, without FCA, was repeated two weeks later. Pre-immunization sera were collected from each fish prior to immunization. Post immunization sera were collected one week after the second injection. Infectious tomites were collected according to the method as previously described (Ling, K. H, Sin, Y. M. and Lam, T. J., “PROTECTION OF GOLDFISH AGAINST SOME ECTOPARASITIC PROTOZOANS USING ICHTHYOPHTHIRIUS MULTIFILIIS AND TETRAHYMENA PYRIFORMIS FOR VACCINATION”, Aquaculture, 116 (1993)303-314).

The tomites and thrombin digested GST-iAgI fusion protein were boiled in SDS sample buffer for 5 min, and analyzed by electrophoresis using an SDS-polyacrylamide gel. The resolved protein was electroblotted onto a 0.2 μm nitrocellulose filter. For immunodetection, the filter was blocked in 0.5% bovine serum albumin (BSA) in TTBS (0.05% Tween- 20, 10 mM Tris-HCl, 150 mM NaCl, pH7.5) at 4° C. for 16 hours, followed by incubating with 1:50 primary antibody (catfish antisera against GST-iAgI fusion protein) overnight at 4° C. After three washes with TTBS, the filter was incubated with 1:100 of rabbit anti-catfish serum for 1 hour and then with 1:3,000 dilution of goat anti-rabbit IgG alkaline phosphate conjugates for 1 hour (Bio-Rad). The filter was washed with TTBS and incubated with substrate NBT (nitroblue tetrazolium ) and BCIP(5-bromo-4-chloro-3-indoyl phosphate) for color development at room temperature.

Challenge Test With Infectious Tomites

Goldfish, Carassius auratus (L.), weighing 5-6 gms, were used throughout the study. All fish were bred from healthy broodstock and were not previously infected by I. Multifiliis nor by any other obvious parasites. The fish were maintained in the laboratory as described previously (Ling, K. H., Sin, Y. M. and Lam, T. J., “A NEW APPROACH TO CONTROLLING ICHTHYOPHTHIRIASIS IN A CLOSED CULTURE SYSTEM OF FRESHWATER ORNAMENTAL FISH”, J. Fish Dis., 14 (1991) 595-598).

Each fish was immunized by intraperitoneal injection of 15 μg GST-iAgI fusion protein with FCS and boosted, by the injection of a similar dose of GST-iAgI fusion protein without the adjuvant, two weeks later. The control group was similarly injected with PBS and FCA mixture at the first injection, and then boosted with PBS alone. One week after the last injection, the control and immunized fish were challenged by direct exposure to infectious tomites using the method of Ling, K. H., Sin, Y. M. and Lam, T. J., “A NEW APPROACH TO CONTROLLING ICHTHYOPHTHIRIASIS IN A CLOSED CULTURE SYSTEM OF FRESHWATER ORNAMENTAL FISH”, J. Fish Dis., 14(1991) 595-598. Each fish of all groups was exposed to 5,000 tomites. The fish were kept in aerated water at room temperature for four weeks under observation for infection and death. The degree of infection was separated and recorded in three (3) categories: none (free of parasites), mild, and heavy infection. Fish infected with less than 100 parasites per fish, and had parasites only on their fins were considered to have a mild infection. Those infected with more than 100 parasites, and not only on their fins but also on their body surface, were considered to have a heavy infection. The significance of the survival rate of fish between the immune and the control group after challenge was analysed by Chi-square test.

### SPECIFIC EXAMPLES AND RESULTS

### Example 1

The amino acid sequence of i-AgI (Clark, T. G., McGraw, R. A. and Dickerson, H. W., “DEVELOPMENTAL EXPRESSION OF SURFACE ANTIGEN GENES IN THE PARASITIC CILIATE ICHTHYOPHTHIRIUS MULTIFILIIS”, Proc. Natl. Acad. Sci. USA, 89 (1992) 6363-6367) was analyzed by a computer program to determine if it had a potential antigenic epitope, Predict 7 V1.01© (Carmenes, R. S., Freije, J. P., Molina, M. M. and Martin, J. M., “PREDICT 7, A PROGRAM FOR PROTEIN STRUCTURE PREDICTION”, Biochem. Biophys. Res. Commun., 159 (1989) 687-693). It was predicted that each of the three repeats could be a strong antigenic epitope. The repeat I (FIG. 1A) was selected for expression of the recombinant protein for immunization.

Three pairs of oligonucleotides were designed and synthesized to cover the repeat I of i-AgI (see FIG. 1). For the expression of i-AgI recombinant protein , the assembled gene fragment was inserted into the bacterial expression vector pGEX2T and the resulting plasmid was named pGST-iAgI (see FIG. 1). As shown in FIG. 2(a), the expression of GST-iAgI fusion protein can be induced after addition of IPTG (lane 4). The GST-iAgI fusion protein can be purified by glutathione Sepharose 4B bead (lane 6), and the purified GST-iAgI fusion protein can be cleaved at the fusion site with thrombin (lane 5). The yield of GST-ich fusion protein, purified from 1 liter bacterial culture, was 2.7-3.0 mg.

### Example 2

To test whether the recombinant protein contained the antigenic epitope for i-AgI, antisera against GST-iAgI, fusion protein was raised in catfish and tested for immunological reaction with the total tomites' proteins. As shown in FIG. 2(b), the catfish antisera reacted strongly to the intact fusion protein, as well as to the GST and the iAgI portions after thrombin digestion (lane 1). It is interesting to note that the catfish antisera also reacted with a tomite's protein of a molecular weight of about 48K (lane 2). This is consistent with the predicted molecular weight of i-AgI. All the sera obtained from fish prior to immunization were negative (data not shown).

### Example 3

To test the effectiveness of the GST-iAgI as a vaccine, naive goldfish were immunized with the fusion protein and then challenged with infectious tomites of I. Multifiliis. Clinical signs of the disease and mortalities of the fish were recorded from day 4 onwards after the exposure to these tomites. The result are summarized in Table 1. 22.7% of the immunized fish showed no clinical sign of the disease, while 50% of the immunized fish exhibited heavy infection, and 27.3% showed a mild infection. In contrast, all the control fish were infected: 75% of non-immunized fish were heavily infected, and the other 25% were mildly infected. The average survival rate of the immunized fish was 95% as compared to 55% for the control. The difference between the survival rate of the control and of the immunized group is significant, p<0.001 as determined by Chi-square-sequare test.

TABLE 1
Infectivity and average survival rate of control and immunized fish
after being exposed to infective tomites of I. Multifiliis.

| | | Degree of | | | |
|---|---|---|---|---|---|
| | | infectivity (%)** | | | |
| Group | Total fish* | heavy | mild | none | Survival (%)*** |
| Control | 20 | 75 | 25 | 0 | 55% |
| Immune | 22 | 50 | 27.2 | 22.8 | 95% |

*Two duplicate groups of 10 to 12 fish each.
**Heavy: the number of parasites found on the fish body and fins was more than 100 up to 500-1000;
Mild: the number of parasites found only on the fins was less than 100; None: the fish were free of parasites
***significantly different at p < 0.001 level, between control and immunized fish by the Chi-square test

Further Application of The Recombinant Vaccine

The effectiveness of this recombinant vaccine in immune protection against Ichthyophthirius multifiliis has been demonstrated in fish (He, J., Yin, Z, Xu, G., Gong Z., Lam T. J. and Sin Y. M., “PROTECTION OF GOLDFISH AGAINST ICHTHYOPHTHIRIUS MULTIFILIIS BY IMMUNIZATION WITH A RECOMBINANT VACCINE”, Aquaculture, 158 (1997)1-10). The antigenicity of the vaccine was also shown using antisera against GST fusion protein from catfish that reacted strongly to the protein of I. Multifiliis of a similar size (48 KDa) to that of the immobilization antigen of I. Multifiliis (i-AgI) as reported by Clark, T. G., McGraw, R. A. and Dickerson, H. W., “DEVELOPMENTAL EXPRESSION OF SURFACE ANTIGEN GENES IN THE PARASITIC CILIATE ICHTHYOPHTHIRIUS MULTIFILIIS”, Proc. Natl. Acad. Sci. USA, 89 (1992) 6363-6367). In fact, fish immunized with the immobilization protein of I. Multifiliis and T. pyriformis showed protection against the disease caused by I. Multifiliis in freshwater fish (Goven , B. A., Dawe, D. L. and Gratzek, J. B., “PROTECTION OF CHANNEL CATFISH, ICTALURUS PUNCTATUS RAFINESQUE, AGAINST ICHTHYOPHTHIRIUS MULTIFILIIS FOUQUET BY IMMUNIZATION”, J. Fish Biol., 17 (1980) 311-316; Goven,B. A., Dawe, D. L and Gratzek, J. B., “IN VITRO DEMONSTRATION OF SEROLOGICAL CROSS-REACTIVITY BETWEEN ICHTHYOPHTHIRIUS MULTIFILIISFOUQUET AND T. PYRIFORMIS LWOFF”, Dev. Comp. Immunol.,5 (1981) 283-289) and Cryptocryon in marine fish (U.S. Pat. No. 4,309,416). The immune protection was correlated with the levels of immobilization antibody in the plasma and skin mucus of the freshwater fish immunized against I. Multifiliis or T. pyriformis (Clark, T. G., Dickerson, H. W., and Findly, R. C., “IMMUNE RESPONSE OF CHANNEL CATFISH TO CILIARY ANTIGENS OF ICHTHYOPHTHIRIUS MULTIFILIIS”, Devel. Comp. Immunol.,12 (1988)581-594; Ling, K. H., Sin, Y. M. and Lam T. J., “PROTECTION OF GOLDFISH AGAINST SOME COMMON ECTOPARASITIC PROTOZOANS USING ICHTHYOPHTHIRIUS MULTIFILIIS AND TETRAHYMENA PYRIFORMIS FOR VACCINATION”, Aquaculture, 116 (1993) 303- 314). An immobilization antigen extracted from T. pyriformis was also shown to be an effective vaccine against I. Multifiliis and Cryptocryon in marine fish (U.S, Pat. No. 4,309,416). Since fish immunized with either I. Multifiliis or T. pyriformis developed protective immunity not only against I. Multifiliis but also against other ciliated ectoparasitic protozoans that are commonly found in the tropics, therefore, the recombinant vaccine made for the immobilization antigen of I. Multifiliis should also exert the same immune protection not only against I. Multifiliis but also against other taxonomically related ciliated ectoparasitic protozoans in the freshwater and the marine fish.

For immunization by injection, the fusion protein was dissolved in buffer and mixed with Freund's complete adjuvant (FCA). Any substance or medium that does not chemically destroy or interfere with the immunogenicity of the fusion protein can be incorporated into the formulation of the vaccine. These include, for example, buffers, immunostimulants and carriers. The vaccine can be introduced to fish by intraperitoneal injection, incorporataed into fish diet, and by immersion or by spraying of the fish. For injection of a 5-6 gm fish, at least about 15 μg of the fusion protein is required. That is, a dose rate of at least about 3 μg fusion protein per gram of fish is appropriate

SEQ. ID NO 1
GA/SAQ*GEANGNQ*PFAANNAARGI[CVPCQINRVGSV/GTNAGD
LATLATQ*CSTQ*CPTGTALDDGVTDVFDRSAAQ*CVKCKPNFY
YNGGSPQ*GEAPGVQ*VFAAGAA]AAGV/I*
SEQ. ID NO 2
GGT GCT GCT TAA GGA GAA GCT AAT GCT AAT TAA CCT
TTC GCA GCA AAT AAT GCT GCT AGA GGT ATA TGT GTA
CCA TGC CAA ATA AAC AGA GTA GGC TCT GTT ACC AAT
GCA GGT GAG TTA GCT ACT TTA GCC ACA TAA TGC AGT
ACT TAA TGT CCT ACT GGC ACT GCA CTT GAT GAT GGA
GTG ACA GAT GTT TTT GAT AGA TCA GCC GCA TAA TGT
GTT AAA TGC AAA CCT AAC TTT TAC TAT AAT GGT GGT
TCT CCT TAA GGT GAA GCT CCT GGC GTT TAA GTT TTT
GCT GCT GGT GCT GCC GCT GCA GGT GTT G
SEQ. ID NO 3
GGA TCC GCT CAG GGA GAA GCT AAT GGT AAT CAG CCT
TTC GCA GCA AAT AAT GCT GCT AGA GGT ATA TGT GTA
CCA TGC CAA ATA AAC AGA GTA GGC TCT CGT ACC AAT
GCA GGT GAC TTA GCT ACT TTA GCC ACA CAA TGC AGT
ACT CAG TGT CCT ACT GGC ACT GCA CTT GAT GAT GGA
GTG ACA GAT GTT TTT GAT AGA TCA GCC GCA CAG TGT
GTT AAA TGC AAA CCT AAC TTT TAC TAT AAT GGT GGT
TCT CCT CAG GGT GAA GCT CCT GGC GTT CAG GTT TTT
GCT GCT GGT GCT GCC GCT GCA CGA ATT C

What is claimed is:

1. A vaccine for immunizing fish against ciliated ectoparasitic protozoans comprising an effective amount of a fusion protein expressed from a recombinant DNA sequence for immobilization antigen, repeat I of Ichthyrophthrius multifiliis, wherein said fusion protein incorporates SEQ. ID. No. 9 or 10 therein or both.

2. The vaccine of claim 1, wherein the fusion protein is GST-iAgI.

3. The vaccine of claim 1, further comprising a medium comprising at least one of a buffer, antigen, immunostimulant or carrier.

4. The vaccine of claim 1, which, when injected into a fish, provides effective protection against white spot disease caused by Ichthyophthirius multifiliis.

5. The vaccine of claim 1, wherein, the fusion protein is produced by E. coli.

6. The vaccine of claim 4, wherein the disease is caused by at least one ciliated ectoparasitic protozoan.

7. The vaccine of claim 6, wherein said ciliated ectoparasitic protozoan is Ichthyophthirius multifiliis.

8. The vaccine of claim 6, wherein said ciliated protozoan is taxonomically related to Ichthyophthirius multifilii.

9. The vaccine of claim 1, wherein said fusion protein incorporates SEQ. ID. No. 9 therein.

10. The vaccine of claim 2, wherein the fusion protein is produced by E. coli containing plasmid construct pGST-iAgI.

11. The vaccine of claim 1, wherein the fusion protein is purified by glutathione sepharose 4B beads.

12. The vaccine of claim 1, which further comprises Freund's complete adjuvant (FCA), and which vaccine is suitable for administration by injection.

13. The vaccine of claim 1, which is in a form suitable for administration by intraperitoneal injection, incorporation into fish diet, or immersion or spraying of the fish.

14. The vaccine of claim 1, in a single dose form, whereby about 3 g of fusion protein per gram of fish treated is provided therein.

15. The vaccine of claim 1, wherein said fusion protein incorporates SEQ. ID. No. 10 therein.

16. The vaccine of claim 1, wherein the recombinant DNA sequence further comprises synthetic oligonucleotides .

17. The vaccine of claim 16, wherein the synthetic oligonucleotides are selected from the group consisting of SEQ. ID. Nos. 11-16.

18. The vaccine of claim 1, wherein the fusion protein is isolated and purified prior to inclusion in the vaccine.
